(19) United States
(12) Patent Application Publication
(10) Pub. No.: US 2021/0346524 A1
(43) Pub. Date: Nov. 11, 2021

(54) FUSOGENIC LIPOSOMES FOR SELECTIVE IMAGING OF TUMOR CELLS

(71) Applicant: APA- Advanced Technologies Ltd., Tel-Aviv (IL)

(72) Inventors: Igor NUDELMAN, Tel-Aviv (IL); Galoz KANETI, Tel-Aviv (IL); Ruslana MILITSIN, Tel-Aviv (IL); Avi GOLDSOBEL, Tel-Aviv (IL); Avi SCHROEDER, Tel-Aviv (IL); David GERSHON, Tel-Aviv (IL); Haim ALCALAY, Tel-Aviv (IL)

(21) Appl. No.: 17/240,117

(22) Filed: Apr. 26, 2021

Related U.S. Application Data
(63) Continuation-in-part of application No. PCT/IL2019/051153, filed on Oct. 24, 2019.
(60) Provisional application No. 62/749,794, filed on Oct. 24, 2018.

(51) Int. Cl. A61K 49/18; A61K 49/00; A61K 47/69; G01N 33/542; A61B 5/00; A61N 5/10

(52) U.S. Cl. CPC A61K 49/1812; A61K 49/0086; A61K 47/6913; A61B 2090/3941; G01N 33/542; A61B 5/0071; A61N 5/1049; A61K 47/6915

(57) ABSTRACT

A fusogenic liposome comprising a detectable agent and optionally a cytotoxic drug in its internal aqueous compartment or bound to the liposome membrane is provided, wherein said fusogenic liposome comprises a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; and at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide. Methods utilizing these liposomes in treatment of cancer are further provided.

The Sequence Listing in ASCII text file format of 1,385 bytes in size, created on Jun. 29, 2021, with the file name “2021-06-30SequenceListing-NUDELMAN7A,” filed in the U.S. Patent and Trademark Office on Jun. 30, 2021, is hereby incorporated herein by reference.

### CROSS-REFERENCE TO RELATED APPLICATIONS

The present application is a continuation-in-part of International Application Number PCT/IL2019/051153, filed Oct. 24, 2019, which claims benefit of Provisional Application U.S. 62/749,794, filed Oct. 24, 2018. The entire contents of each of the above-identified applications are hereby incorporated herein by reference.

### FIELD OF THE INVENTION

The present invention relates in general to methods for selectively detecting cancer cells.

### BACKGROUND OF THE INVENTION

Fluorescence detection (WO 03/057259, U.S. Pat. Nos. 8,956,591, 9,289,517), nuclear magnetic resonance imaging (MRI), Computer tomography (CT), and ultrasonographic imaging are commonly used as methods of imaging a cancer lesion. However, these techniques label the site of a tumor indirectly and do not facilitate selective labeling of cancer cells per se. There therefore remains an urgent need for methods of selective cancer cell imaging.

### SUMMARY OF INVENTION

PCT application No. PCT/IL2018/050434 (incorporated by reference as if fully disclosed herein) discloses fusogenic liposomes and their use in treating cancer. The cancer-selective fusogenic liposome can be administered systemically, and is shown herein to fuse with cancer cells under in vivo conditions. This cancer selective liposome can further be used to carry detectible moieties to cancer sites in the body. The inherent ability of these liposomes to selectively fuse with cancer cells (and not with normal cells) is utilized in this invention for selective detection of cancer cells.

In one aspect, the present invention provides a fusogenic liposome comprising a detectable agent and optionally a cytotoxic drug in its internal aqueous compartment or bound to the liposome membrane, wherein

said fusogenic liposome comprises a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; and

at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide.

In an additional aspect, the present invention provides a method for selectively detecting cancer cells, comprising contacting said cancer cells with a fusogenic liposome defined herein; and in case said detectable agent is an activatable fluorescent probe, detecting said fluorescent probe by illuminating the cell with light having a wave length that is absorbed by the fluorescent probe and detecting light emitted from the excited fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent.

In a further aspect, the present invention provides a method for selectively detecting cancer cells, comprising (a) contacting said cancer cells with a functionalised fusogenic liposome according to any one of the above embodiments directed to functionalised fusogenic liposomes; (b) contacting said cancer cells with a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) in case said detectable agent is a fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby selectively detecting said cancer cells.

In yet an additional aspect, the present invention provides a method for treating cancer by fluorescence-guided surgery or targeted radiotherapy said method comprising one of the methods for selectively detecting cancer cells defined above and removing the tumor containing the cancer cells.

In certain embodiments of any one of the methods for selectively detecting cancer cells defined above, the cancer patient is undergoing imaging of tumors, such as skin cancer, or whole body imaging, and the method or use comprises systemically administering or topically applying the fusogenic liposome, and optionally the functionalized detectable agent; and, in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device scanning an area of an organ, such as skin, or whole body, thereby defining tumor location and margins.

In yet a further aspect, the present invention provides a method for treating cancer comprising the method in which the cancer patient is undergoing imaging of tumors, such as skin cancer, or whole body imaging described above, and surgically removing the tumor containing the cancer cells.

In yet another aspect, the present invention provides a kit comprising: (a) a first container comprising a fusogenic liposome comprising a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide; and wherein at least one of said lipid molecules is functionalised with a first functional group of a specific binding pair capable of binding to a complementary second functional group of said binding pair; (b) a second container comprising a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) a pamphlet with instructions for a method for selectively detecting cancer cells comprising administering to a cancer patient the fusogenic liposome of (a) and subsequently the detectable agent of (b).

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1D show photographs of in situ detection of triple negative breast cancer in mice. 24 hrs post IV injection of ICG loaded liposomes, tumor bearing mice were imaged using IVIS® Spectrum in vivo imaging system (PerkinElmer). Tumor signal (mCherry; FIGS. 1A and 1C) and liposome signal (ICG; FIGS. 1B and 1D) was recorded. Liposomes injected to mice shown in FIGS. 1A and 1B images were modified using PEG₄-azide and mice shown in FIGS. 1C and 1D were injected with liposomes modified with PEG₄-Biotin.

FIGS. 2A-2C show fluorescent and non-fluorescent images overlay image of organs at 48 hrs post injection. 48 Hrs post IV injection of ICG loaded liposome, mice were sacrificed, organs were removed and imaged under identical conditions as in FIGS. 1A-1D (excitation and emission) to determine ICG signal from liposomes. Organ order top to bottom, left to right: Lungs, Liver, Spleen, Kidneys, Tumor. Untreated mouse (FIG. 2A) was used to determine threshold levels. Organs from three mice from Azide modified liposomes (FIG. 2B) and from Biotin modified liposomes (FIG. 2C) show fluorescent signal at tumors and livers.

FIGS. 3A-3C show fluorescent signal change in tumors of tumor-bearing mice, compared with tumor arrival of Gd-DTPA and biodistribution profiles at 24 Hrs post last injection. Gadolinium (Gd) and Indocyanine green (ICG) co-loaded liposomes, bound to T cell activating antibodies (anti-CD3 and anti-CD8) were injected to tumor-bearing mice to test tumor site arrival at 2nd treatment to determine optimal interval between treatments. This proof of concept compares delivery of MRI contrast agent, Gd-DTPA with fluorescent probe ICG. FIG. 3A. Tumor Gd levels were determined using ICP analysis at 335.048 nm emission. Single treatment was compared with second treatment at: 48 Hrs (vertical lines), 72 Hrs (diagonal rectangles,) and 96 Hrs (horizontal lines) N=4 animals per group. FIG. 3B. IVIS Spectrum CT in-vivo imaging system was used to record mCherry (tumor) and ICG (liposome) signals. Manual tumor region of interest was determined using mCherry signal and was used to record ICG photons per second. Data presented is ratio of second treatment versus single treatment. Error bars represent standard deviation. FIG. 3C. Biodistribution of liposomes as determined by ICP is presented as percent injected dose per gram tissue. Tumor liposome arrival is compared with liver (horizontal line, spleen (horizontal rectangles), kidney (vertical lines) heart (diagonal lines) and lungs (diagonal rectangles) in single or two treatments at different intervals.

FIGS. 4A-4D shows clearance of liposomes and their accumulation in treated mice versus untreated mice using ICG loaded liposomes. FIG. 4A. 4T1 mCherry tumor bearing mice were imaged using IVIS® Spectrum in vivo imaging system (PerkinElmer) to determine mCherry signal emitted from tumors (ex 570 nm, em 640 nm, exposure time 1 sec) on the day of the treatment (left mouse and right mouse). FIG. 4B. mice (first and fourth mouse from left) were treated with ICG- and Gd-DTPA co-encapsulated with therapeutic antibodies bound to liposome (anti-CD3 and anti-CD8) and were imaged using IVIS® Spectrum in vivo imaging system (PerkinElmer) to determine liposomal ICG signal emitted from entire animal body (ex 745 nm, em 870 nm, exposure time 4 sec) on the day of the treatment. The 2^{nd} and 3^{rd} mouse from left did not receive ICG- and Gd-DTPA liposomes. FIG. 4C. Presented mCherry signal emitted from tumors, same as FIG. 4A, at 24 hrs post treatment. FIG. 4D. Presented liposomal ICG signal emitted from entire animal body, same as FIG. 4B, at 24 hrs post treatment.

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the recited elements, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It has been found in accordance with the present invention that a fusogenic liposome having the characteristics of preferably fusing with cancer cells over normal cells can be used to selectively detect and visualize cancer cells in vivo or in vitro.

Thus, in one aspect, the present invention provides a fusogenic liposome comprising a detectable agent and optionally a cytotoxic drug in its internal aqueous compartment or bound to the liposome membrane, wherein

said fusogenic liposome comprises a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; and

at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide.

The term “liposome” as used herein refers to a lipid nanoparticle or construct comprising a lipid bilayer composed of an inner and an outer leaflet, which encapsulates an aqueous interior of the liposomes.

The term “fusogenic liposome” as used herein refers to a liposome construct that preferentially fuses with the plasma membrane of a target cell and is taken up by endocytosis to a lesser degree.

In general, as defined herein, the term “labelling (of) cells” relates to any modification of the cells structurally distinguishing them from the unmodified cells. In particular, the cells in the present invention are modified or “labeled” with a functional group of a fusogenic liposome or with a detectable agent.

The term “stabilizing moiety” as used herein refers to a moiety that when incorporated within the lipid bilayer of the liposome provides prolonged blood circulation half-life of the liposomes as compared with an identical liposome lacking the stabilizing moiety.

In certain embodiments, the detectable agent is selected from a fluorescent probe, a contrast agent for magnetic resonance imaging (MRI), computed tomography (CT) or positron emission tomography (PET), and a photodynamic agent.

In certain embodiments, the detectable agent is a fluorescent probe selected from cy3, cy5, cy5.5, cy7 cy9, FITC, fluorescein, alexa fluor 790, alexa fluor 750, alexa fluor 700, alexa fluor 680, alexa fluor 660, alexa fluor 647, alexa fluor 633, alexa fluor 594, Qdots ranging 585 nm to 800 nm, fluorescent protoporphyrin oligomers, isocyanine green (ICG); or an activatable fluorescent probe, a type of activatable ester bond cleavable fluorophore. The ester modification(s) on the fluorophore render it non-fluorescent, and it becomes fluorescent upon fusion of the liposome with a cancer cell and subsequent cytoplasmic cleavage by cytoplasmic esterases.

In certain embodiments the activatable fluorescent probe is selected from fluorescein analogs (such as di-acetate modified analogs), coumarin analogs (such as py+BC690-(1-Methyl-4-(2-oxo-8-(pyrrolidin-1-yl)-2H-benzo[g]chromen-3-yl)pyridinium trifluoromethanesulfonate)), CFSE (5(6)-Carboxyfluorescein diacetate N-succinimidyl ester), rhodamine analogs (such as gGlu-HMRG (γ-glutamyl hydroxymethyl rhodamine green)), curcuminoid difluoroboron-based tumor-targeting γ-glutamyltranspeptidase (GGT)-activatable) fluorescent probe (Glu-DFB), indocyanine analog (such as AP-Glu (3H-Indolium, 2-[(1E)-2-[4-[[4-[[(4S)-4-amino-4-carboxy-1-oxobutyl]amino]phenyl]methoxy]phenyl] ethenyl]-1-(5-carboxypentyl)-3,3-dimethyl-, bromide (1:1), CAS Registry Number 1884698-06-9) and other near-infrared fluorescence activated molecule.

Under in vivo conditions far red fluorophores (630/661 or longer wavelengths; such as Alexafluor633, cell trace far-red, or Cy5) are preferred since tissue autofluorescence is relatively low, and long wavelength tissue penetration is better than shorter ones.

In certain embodiments, the contrast agent for MRI is selected from iron oxide contrast agents (such as magnetite, Fe₃O₄); barium sulfate; and gadolinium contrast agents, such as gadoterate, gadodiamide, gadobenate, gadopentetate, gadobutrol; the contrast agent for CT is selected from metal elements, such as iodine, bismuth, bromine, tantalum, gold, platinum, ytterbium, yttrium, gadolinium, tungsten, indium and lutetium; or the contrast agent for PET is selected from ⁶⁴Cu-PSTM, ¹⁸F-FDG, ¹⁸F-fluoride, ¹⁸F-fluoromisonidazole and Gallium.

In certain embodiments, the photodynamic agent is selected from Porfimer sodium (Photofrin®), Metvix®/Metvixia, temoporfin/mTHPC/Foscan, talaporfin/NPe6/Laserphyrin, Redaporfin®/LUZ11, Tookad, Photochlor, Fotolon, Antrin, Purlytin, TLD1433, WST11, and Lutex, or gold nanoparticles such as sphereshape or road shape nanoparticles.

In certain embodiments, the hydrophilic head of the at least one lipid of the plurality of lipids is each functionalised with a first functional group or a second functional group of a binding pair capable of binding to each other under normal conditions in preference to binding to other molecules or forming between themselves a covalent bond or non-covalent high-affinity conjugate, wherein the first functional group and the second functional group of the binding pair is for example, but is not limited to, (i) reactive groups of a click chemistry reaction; or (ii) a biotin and a biotin-binding peptide or biotin-binding protein.

The term “high affinity” as used herein refers to a chemical or bio-physical association, such as chelator-metal coupling (e.g. Ni and a peptide sequence comprising several His-residues such as His₆), or an conjugation between two members of a binding pair, e.g. an antibody and its target epitope or biotin and streptavidin, etc., wherein the association between two binding pairs has a K_{d} of 10⁻⁴ M to 10⁻³⁰ M, e.g. 10⁻⁶ M, 10⁻⁷ M, 10⁻⁸ M, 10⁻⁹ M, 10⁻¹⁰ M, 10⁻¹¹ M, 10⁻¹² M or 12⁻¹³ M.

In certain embodiments, the at least one of said lipid molecules is functionalised with a first functional group of a specific binding pair capable of binding to a complementary second functional group of said binding pair.

The term “binding pair” as used herein refers to a pair of different molecules, each comprising its own specific functional group, both functional groups have particular specificity for (or complimentary to) each other. In other words, these groups, under normal conditions, are capable of binding to each other in preference to binding to other molecules. The binding may be covalent or non-covalent. Non-limiting examples of such binding pairs are thiol-maleimide, azide-alkyne, aldehyde-hydroxylamine etc.

In general, a functional group is a specific group or moiety of atoms or bonds within molecules that is responsible for the characteristic chemical reactions of those molecules. In particular, a functional group, or a functional group of a binding pair, as defined herein, refers to a specific reactive group or moiety of atoms or bonds of the binding pair (hereinafter “a first functional group”) capable of binding to another functional group of said binding pair (hereinafter “a second functional group”). As mentioned above, the first and the second functional groups are complementary to each other. In the above non-limiting examples, the first functional groups are thiol, azide or aldehyde and their complementary (second) functional groups are maleimide, alkyne or hydroxylamine, respectively.

In general, crosslinking reagents (or crosslinkers) as defined herein refer to molecules that contain two or more reactive ends (functional groups) capable of chemically attaching to specific reactive groups (primary amines, sulfhydryls, etc.) on proteins or other molecules. In particular, the crosslinkers as defined herein comprise functional groups and spacers.

In certain embodiments, the fusogenic liposome further comprises a first spacer between the lipid bilayer and the first functional group.

In certain embodiments, the fusogenic liposome at least one of said lipid molecules functionalised with a first functional group further comprises an identical or different additional detectable agent, phototherapeutic agent, i.e. photodynamic agent, or an immune system activating agent, each one functionalised with said complementary second functional group and bound to said first functional group via said second functional group, wherein said additional identical or different detectable agent is selected from a fluorescent probe and a contrast agent for magnetic resonance imaging (MRI), computed tomography (CT) or positron emission tomography (PET).

In certain embodiments, the immune system activating agent is agent is selected from anti-CD3 antibody, an anti-CD8 antibody, an anti-NKG2D antibody, or a combination thereof, an antibody capable of binding both CD3 and CD8 and an antibody capable of binding both CD3 and NKG2D.

When the fusogenic liposome comprises an immune system activating agent or phototherapeutic agent such as gold nanoparticles it is essentially a Theranostics agent.

In certain embodiments, the detectable agent, phototherapeutic agent, or immune system activating agent is bound at the outer leaflet of the fusogenic liposome.

In certain embodiments, the detectable agent or immune-system activating agent further comprises a second spacer between the detectable agent or immune-system activating agent and the second functional group.

In certain embodiments, the first or second spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide, such as a poly-Gly peptide of about 2-20 amino acid residues in length, e.g. 3 amino acid residues in length.

In certain embodiments, the first or second spacer is PEG of molecular weight of about 106 Da to about 4 kDa.

In certain embodiments, the first or second spacer is PEG of a molecular weight of about 194 Da (PEG₄).

In certain embodiments, the first or second spacer is (C₆-C₁₂)alkyl, preferably heptyl or dodecanoyl.

In certain embodiments, the immune-system activating agent is selected from a T-cell activating agent; a pro-inflammatory cytokine; a memory killer T cell activating peptide; a soluble human leukocyte antigen (sHLA) presenting a peptide; and a super-antigen.

In certain embodiments, the immune-system activating agent is a T-cell activating agent.

In certain embodiments, the T-cell activating agent is selected from anti-CD3 antibody, an anti-CD8 antibody, an anti-NKG2D antibody, or a combination thereof, an antibody capable of binding both CD3 and CD8 and an antibody capable of binding both CD3 and NKG2D.

The antibodies or functional fragments thereof described herein refer also to a single chain variable fragment (scFv); a functional fragment of an antibody; a single-domain antibody, such as a Nanobody; and a recombinant antibody; (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer.

It should be made clear that the antibodies or functional fragments thereof used in the present invention do not fulfil the function of targeting agent (to bring the liposome to a certain target cell), but instead fulfil the function of immune system activating agent.

In any one of the above embodiments, the at least one of said lipid molecules comprising a cationic group is selected from 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), Dimethyldioctadecylammonium (18:0 DDAB), and N1-[2-((1S)-1-[(3-aminopropyl)amino]-4-[di(3-amino-propyl)amino]butyl-carboxamido)ethyl]-3,4-di[oleyloxy]-benzamide (MVL5).

In certain embodiments, the at least one of said lipid molecules comprising a cationic group is DOTAP.

In any one of the above embodiments, the cationic synthetic polymer is selected from polyethyleneimines (PEI) and poly(2-(dimethylamino)ethyl methacrylate.

In any one of the above embodiments, the cationic natural polymer is chitosan.

In any one of the above embodiments, the cationic amino sugar is glucosamine.

In any one of the above embodiments, the cationic polyamino acid is selected from poly(L-lysine), poly(L-arginine), poly(D-lysine), poly(D-arginine), poly(L-ornithine) and poly(D-ornithine).

In any one of the above embodiments, the amphiphilic cancer-cell binding peptide is selected from Cecropin A; Cecropin A 1-8; and cyclic CNGRC.

In any one of the above embodiments, the at least one of said lipid molecules is a phospholipid selected from the group consisting of a phosphatidylcholine, a phosphatidylethanolamine, a phosphatidylserine, a phosphatidic acid or a combination thereof, each one of which comprises one or two identical or different fatty acid residues, wherein the fatty acid residues in the phosphatidyl moiety is saturated, mono-unsaturated or poly-unsaturated and has a carbon chain length of 14, 15, 16, 17, 18, 19, 20, 21, 22, 23 or 24 carbons, such as myristoyl, stearoyl, palmitoyl, oleoyl, linoleoyl, linolenoyl (including conjugated linolenoyl), arachidonoyl in phospholipid and lyso-phospholipid configuration, and combinations thereof.

In certain embodiments, the phospholipid is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-distearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (Δ9-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-distearoyl-3-phosphatidylethanolamine (DSPE); 1,2-dimyristoyl-3-phosphatidylserine (DMPS); 1,2-dipalmitoyl-3-phosphatidylserine (DPPS); palmitoyloleoyl phosphatidylethanolamine (POPE); and 1,2-dioleoyl-3-phosphatidylserine (DOPS).

In certain embodiments, the phospholipid is selected from DOPC, POPC, DMPC, DPPC, DOPE, POPE, DSPE, DMPE and DPPE.

In any one of the above embodiments, the stabilizing moiety is PEG of molecular weight of about 106 Da to about 4 kDa.

In certain embodiments, the PEG of the stabilizing moiety is of molecular weight of about 2 kDa.

In any one of the above embodiments, the stabilizing moiety is connected to at least one of said lipid molecules via a cleavable peptide linker.

In any one of the above embodiments directed to liposomes comprising functional groups, the first functional group of the specific binding pair is capable of forming a covalent bond with said complementary second functional group of said binding pair.

In certain embodiments, the first functional group of the specific binding pair is capable of forming a covalent bond with said complementary second functional group of said binding pair via a click chemistry reaction.

In certain embodiments, i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa; iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or derivative thereof such as ester, anhydride, acyl halide, tosyl and N-hydrosuccinimide (NHS), and the second functional group of said binding pair is amine, or vice versa; viii) functional group.

In certain embodiments, the specific binding pair is alkyne-azide.

In any one of the above embodiments directed to liposomes comprising functional groups, the first functional group of the specific binding pair is capable of forming a non-covalent bond with said complementary second functional group of said binding pair.

In certain embodiments, the first functional group of the specific binding pair is biotin, and the second functional group of said binding pair is its binding-partner selected from a biotin-binding peptide or biotin-binding protein, or vice versa.

In certain embodiments, the biotin-binding protein is selected from avidin, streptavidin and an anti-biotin antibody.

In certain embodiments, the biotin-binding peptide is selected from AEGEFCSWAPPKASCGDPAK (SEQ ID NO: 1), 2 (SEQ ID NO: 2), CSWAPPFKASC (SEQ ID NO: 3), and CNWTPPFKTRC (SEQ ID NO: 4).

In any one of the above embodiments, the fusogenic liposome further comprises cholesterol (CHO) or its derivatives.

In any one of the above embodiments, the fusogenic liposome comprises DOPC:DOTAP:DSPE-PEG2K:DOPE or DOPC:DOTAP:DSPE-PEG2K, and optionally cholesterol, wherein PEG2K represents PEG having a molecular weight of about 2 kDa, and the relative molar amount of DOPC is up to about 80%, the relative molar amount of DOTAP is up to about 80%, the relative molar amount of DSPE-PEG2K is up to about 20%, the relative molar amount of DOPE is up to about 20%, the relative molar amount of cholesterol is up to about 40%.

In certain embodiments, the fusogenic liposome comprises:
(i) DOPC:DOTAP:DSPE-PEG2K:DOPE in the molar ratio 52.5:35:0.6:10, 52.5:35:1.25:10, 52.5:35:2.5:10, 52.5:35:5:10, 52.5:35:0.6:5, 52.5:35:1.25:5, 52.5:35:2.5:5, 52.5:35:5:5, 65:20:5:10, 50:35:5:10, 52.5:35:1.25:7, 52.5:35:1.25:5, or 52.5:35:2.5:7; or
(ii) DOPC:DOTAP:DSPE-PEG2K, in the molar ratio 52.5:35:0.6, 52.5:35:1.25, 52.5:35:2.5, 52.5:35:5, 52.5:35:0.6, 52.5:35:1.25, 52.5:35:2.5, 52.5:35:5, 65:20:5, 50:35:5, 52.5:35:1.25, 52.5:35:1.25, or 52.5:35:2.5.

In certain embodiments, the fusogenic liposome comprises DOPC:DOTAP:DSPE-PEG2K:DOPE in the molar ratio 52.5:35:2.5:5; or DOPC:DOTAP:DSPE-PEG2K, in the molar ratio 52.5:35:2.5.

In any one of the above embodiments, the melting temperature (Tm) of the liposome is below 45° C., at which the fusogenic liposome is maintained at a non-crystalline transition phase thereby providing membrane fluidity required for fusion of liposome with cell membranes.

In any one of the above embodiments, the fusogenic liposome has a size of up to 200 nm, e.g. from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, e.g. about 100 nm.

The methods used for producing the fusogenic liposome of the present invention are based on the concept of a kinetic reaction control. The liposomes are self-assembled from lipid bilayers at much higher reaction rate than the chemical bond is formed between two functional groups. Thus, an unreacted detectable agent and other reagents or catalysts, such as copper catalyst for the copper-dependent click-chemistry reaction, are encapsulated within the aqueous interior of the liposome before any significant chemical reaction occurs in the solution. The detectable agent and/or other reagents needed for the chemical reaction are not encapsulated inside the liposome are further physically removed from the solution, for example by washing the formed liposomes. Non-limiting examples of catalysts for the click chemical reaction to form the liposomes of the present invention are copper (II) acetylacetonate, copper (I) isonitrile and any other active copper (I) catalyst generated from copper (I) salts or copper (II) salts using sodium ascorbate as the reducing agent. The immune system activating agent and other reagents or catalysts may be removed by e.g. dialysis or gel filtration or by reacting one or both of the functional groups of the immune activating agent or lipids with an excess of a corresponding free functional group which depletes the functional groups of the immune activating agent or lipids and thus, stops or inhibits the reaction.

Methods of preparing liposomes are well known in the art Batzri, S. & Korn, E. D. Single bilayer liposomes prepared without sonication. Biochimica et Biophysica Acta (BBA)-Biomembranes 298, 1015-1019 (1973). For example, a lipid solution in an organic solvent may be injected into an aqueous solution having a temperature above the Tm at conditions leading to formation of liposomes e.g. by the means of a nano-assembler assembler or other similar devices, thereby producing fusogenic liposomes; or injecting the lipid solution into an aqueous solution having a temperature above the Tm and mixing, thereby obtaining a liposome solution, and extruding the liposome solution through an extruder comprising at least one support and at least one etched membrane having pores with a diameter between 50 and 400 nm.

In an additional aspect, the present invention provides a method for selectively detecting cancer cells, comprising contacting said cancer cells with a fusogenic liposome defined in any one of the above embodiments and in any combination thereof, and in case said detectable agent is an activatable fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent. In certain embodiments, the detecting is performed ex-situ.

Similarly, in an additional aspect, the present invention provides the fusogenic liposome of any one of the above embodiments for use in selectively detecting cancer cells, the use or detecting comprising contacting said cancer cells with a fusogenic liposome defined in any one of the above embodiments and in any combination thereof, and in case said detectable agent is an activatable fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent.

Similarly, in an additional aspect, the present invention provides use of the fusogenic liposome of any one of the above embodiments for the preparation of a composition for selectively detecting cancer cells, the use or detecting comprising contacting said cancer cells with a fusogenic liposome defined in any one of the above embodiments and in any combination thereof, and in case said detectable agent is an activatable fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent.

The term “contacting said cancer cells with a fusogenic liposome” is used herein to describe any scenario in which the fusogenic liposome of the present invention is administered in vitro to cells or organs comprising cancer cells alone or a mixture of cancer and normal cells, or in vivo or in situ to a patient having cancer or to an organ having cancer cells.

In certain embodiments, selective detection of cancer cells in a cancer patient indicates responsiveness of said cancer patient to treatment of cancer with a cancer drug comprised in a nanoparticle by predicting the ability of a nanoparticle comprising said cancer drug to reach a tumour in said cancer patient.

In certain embodiments selective detection of cancer cells in a cancer patient is performed using biopsies from neoplastic suspected tissues after systemic first binding pair bound-liposomal administration (i.e. administration of liposomes functionalized with a first functional group of a binding pair), stained using second functional group bound to enzyme, such as horse radish peroxidase (HRP) or to a fluorescent probe added in addition to standard staining of biopsy stains and/or counterstains such as haematoxylin, eosin, DAPI, fluorescent antibodies, fluorescent Phalloidin.

In certain embodiments selective detection is performed using biopsies from neoplastic suspected tissues after incubating tissue sections with liposomes bound to first binding pair, later stained using second binding pair bound to HRP or to a fluorescent probe added in addition to standard staining of biopsy stains and/or counterstains such as haematoxylin, eosin, DAPI, fluorescent antibodies, fluorescent Phalloidin.

In certain embodiments, the cancer patient determined as responsive is treated with said nanoparticle comprising said cancer drug.

In certain embodiments, when the cancer patient is determined as responsive, the nanoparticle comprising said cancer drug is for use in treatment of the cancer patient.

In certain embodiments, when the cancer patient is determined as responsive, the present invention provides use of the nanoparticle comprising said cancer drug for the preparation of a medicament for treatment of the cancer patient.

In a further aspect, the present invention provides a method for selectively detecting cancer cells, comprising (a) contacting said cancer cells with a functionalised fusogenic liposome according to any one of the above embodiments directed to functionalised fusogenic liposomes; (b) contacting said cancer cells with a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) in case said detectable agent is a fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby selectively detecting said cancer cells.

Similarly, in an additional aspect, the present invention provides the fusogenic liposome of any one of the above embodiments for use in selectively detecting cancer cells, the use or detecting comprising (a) contacting said cancer cells with a functionalised fusogenic liposome according to any one of the above embodiments directed to functionalised fusogenic liposomes; (b) contacting said cancer cells with a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) in case said detectable agent is a fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby selectively detecting said cancer cells.

Also similarly, in an additional aspect, the present invention provides use of the fusogenic liposome of any one of the above embodiments for the preparation of a composition for selectively detecting cancer cells, the use or detecting comprising (a) contacting said cancer cells with a functionalised fusogenic liposome according to any one of the above embodiments directed to functionalised fusogenic liposomes; (b) contacting said cancer cells with a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) in case said detectable agent is a fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby selectively detecting said cancer cells.

Alternatively, instead of contacting said cancer cells with a detectable agent functionalized with a complementary second functional group in step (b), the cancer cells are contacted with a second liposome or nanoparticle comprising in its surface a different detectable agent than the detectable agent of the functionalised fusogenic liposome of (a) and the second functional group. This combination can be used in a two-step injection manner to improve signal to noise and reduce false positive in tissues such as liver and spleen. The first liposome with the first binding pair fuses with cancer cells and adds one fluorophore. The second nano-particle with the second binding pair binds the groups on the cancer cells labelled with first liposome and delivers a second fluorophore. Presence of two signals reduces false positives and additionally can be used to perform Fluorescence resonance energy transfer (FRET). FRET can also be used under in-vivo conditions, where the first fusogenic liposome carries one fluorophore and the first binding pair, and the second liposome/nanoparticle carries the complementary binding pair and a second fluorophore that can be used in FRET and will yield signal if the two liposomes with the two fluorophores come in close contact.

In certain embodiments, the detectable agent is a fluorescent probe selected from cy3, cy5, cy5.5, cy7 cy9, FITC, fluorescein, alexa fluor 790, alexa fluor 750, alexa fluor 700, alexa fluor 680, alexa fluor 660, alexa fluor 647, alexa fluor 633, alexa fluor 594, Qdots ranging 585 nm to 800 nm, fluorescent protoporphyrin oligomers, isocyanine green (ICG); or an activatable fluorescent probe selected from fluorescein analogs (such as di-acetate modified analogs), coumarin analogs (such as py+BC690-(1-Methyl-4-(2-oxo-8-(pyrrolidin-1-yl)-2H-benzo[g]chromen-3-yl)pyridinium trifluoromethanesulfonate)), CFSE (5(6)-Carboxyfluorescein diacetate N-succinimidyl ester), rhodamine analogs (such as gGlu-HMRG (γ-glutamyl hydroxymethyl rhodamine green)), curcuminoid difluoroboron-based tumor-targeting γ-glutamyltranspeptidase (GGT)-activatable) fluorescent probe (Glu-DFB), indocyanine analog (such as AP-Glu (ap-glu 3H-Indolium, 2-[(1E)-2-[4-[[4-[[(4S)-4-amino-4-carboxy-1-oxobutyl]amino]phenyl]methoxy]phenyl]ethenyl]-1-(5-carboxypentyl)-3,3-dimethyl-, bromide (1:1), CAS Registry Number 1884698-06-9) and other near-infrared fluorescence activated molecule.

In certain embodiments, selective detection of cancer cells in a cancer patient indicates responsiveness of said cancer patient to treatment of cancer with a cancer drug comprised in a nanoparticle by predicting the ability of a nanoparticle comprising said cancer drug to reach a tumour in said cancer patient.

In certain embodiments, the cancer patient determined as responsive is treated with said nanoparticle comprising said cancer drug.

In certain embodiments, when the cancer patient is determined as responsive, the nanoparticle comprising said cancer drug is for use in treatment of the cancer patient.

In certain embodiments, when the cancer patient is determined as responsive, the present invention provides use of the nanoparticle comprising said cancer drug for the preparation of a medicament for treatment of the cancer patient.

Non-limiting examples of types of nanoparticles used in or under development for cancer treatment are liposomes; protein-based nanoparticles, such as albumin, ferritin, gelatin, and transferrin; a copolymer such as poly(lactic-co-glycolic acid) (PLGA); polymeric micelles, such as micelles made of triblock copolymers PEG-DiHyd-PLA containing hydrazone bond; gold nanoparticles; and magnetite core nanoparticles covered with polymers compatible with in-vivo use.

Non-limiting examples of types of cytotoxic drugs that can be formulated in nanoparticle are a chemotherapeutic agent, such as alkylating agents (e.g., cyclophosphamide, ifosfamide, melphalan, chlorambucil, aziridines, epoxides, alkyl sulfonates), cisplatin and its analogues (e.g., carboplatin, oxaliplatin), antimetabolitites (e.g., methotrexate, 5-fluorouracil, capecitabine, cytarabine, gemcitabine, fludarabine), toposiomerase interactive agents (e.g., camptothecin, irinotecan, topotecan, etoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, interleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, anti-androgens (e.g., flutamide, casodex), kinase and tyrosine inhibitors (e.g., imatinib (STI571 or Gleevac); gefitinib (Iressa); and erlotinib (Tarceva), amphiphilic cancer-cell binding peptide is selected from Cecropin A; Cecropin A 1-8; and cyclic CNGRC; photodynamic agent such as Photofrin, Gold.

The cancer drug comprised in a nanoparticle described above can thus be any combination of the above recited nanoparticles and cytotoxic drugs.

Non-limiting examples of nanoparticle drugs, i.e. particles containing a cytotoxic drug is Abraxane® (paclitaxel bound to albumin to prolong circulation time and bio-avaliability); and Doxil®, a doxorubicin formulation encapsulated in a liposome using active loading method. These doxorubicin liposomes (comprised of HSPC, cholesterol and DSPE-PEG2000 at 56.6:38.2:5.3 molar ratio) are one of the leading nano-medicines in cancer therapy.

In an alternative scenario, the cancer patient determined as responsive by means of fusogenic liposomes functionalized with a first functional group of a binding pair is treated with said nanoparticle comprising said cancer drug, wherein said nanoparticle is functionalized with the complementary second functional group. For example, Abraxane® in which the albumin part is modified with the second binding pair is injected systemically, or doxorubicin actively loaded into liposomes functionalized with the second binding pair. The functionalized second nanoparticle is thus targeted to the cancer cell which is already labeled or functionalized with the first functional group and binds to it via the second functional group.

In yet an additional aspect, the present invention provides a method for treating cancer by fluorescence-guided surgery or targeted radiotherapy, said method comprising one of the methods for selectively detecting cancer cells defined above and removing the tumor containing the cancer cells.

In certain embodiments, liposomes of the present invention carrying an esterase cleavable fluorophore (activatable fluorophore) used during surgery can be either injected intravenously or used to irrigate tumor tissue and surrounding tissue suspected as neoplastic tissues. The tissue is then washed with saline solution to remove access of unbound liposome and blood to improve signal to noise. Fusion of liposomes with cancer cells results in removal of the ester groups on the fluorophore and allows detection using fluorescent excitation and emission wavelengths corresponding with the fluorophore used.

In certain embodiments of any one of the methods for selectively detecting cancer cells or the fusogenic liposome for use in selectively detecting cancer cells defined above, the cancer patient is undergoing scanning of an area of an organ, such as skin, or whole body imaging of tumors, such as skin cancer, or whole body imaging, and the method or use comprises systemically administering or topically applying the fusogenic liposome, and optionally the functionalized detectable agent; and, in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby defining tumor location and margins.

In yet a further aspect, the present invention provides a method for treating cancer comprising the method in which the cancer patient is undergoing imaging of tumors, such as skin cancer, or whole body imaging described above, and surgically removing the tumor containing the cancer cells.

In certain embodiments of any one of the methods for selectively detecting cancer cells defined above, the method is intended for selectively detecting cancer cells ex situ in a tissue, such as a biopsy, or a blood-derived fraction removed from the cancer patient in need thereof, said method comprising systemically administering or applying the fusogenic liposome, and optionally the functionalized fluorescent probe, to the tissue or blood-derived fraction removed from the cancer patient and detecting said fluorescent probe by illuminating the tissue or blood-derived fraction and detecting light emitted from the fluorescent probe ex situ, thereby selectively detecting said cancer cells.

In certain embodiments, the tissue is skin, said cancer patient is undergoing surgery for removal of skin cancer (Mohs surgery), and said surgery is repeated until the skin tissue has no detectable cancer cells as detected ex situ.

In certain embodiments, a tissue suspected of neoplasia is taken out and washed with a physiological isotonic buffer to remove blood clots, the tissue is immersed with a solution comprising activatable fluorophore liposomes solution, for example at 0.1-50 mM lipids. Liposome treated tissue is washed with PBS or other physiological isotonic solution (such as saline) and is imaged under fluorescent microscope with excitation and emission wavelengths corresponding with fluorophore.

Alternatively, a tissue suspected of neoplasia is taken out and washed with PBS or other buffer to remove blood clots is immersed with a solution comprising fusogenic liposomes of the present invention comprising a first functional group of a specific binding pair capable of binding to a complementary second functional group of said binding pair (such as solution at 5 mM lipids). Liposome-treated tissue is then washed with a physiological isotonic solution and is immersed in a solution comprising a detectable fluorophore comprising the complementary second functional group, washed and imaged under fluorescent microscope with excitation and emission wavelengths corresponding with fluorophore.

In certain embodiments, the method intended for selectively detecting cancer cells ex situ, is used for selectively detecting circulating tumour cells (CTCs) in said blood-derived fraction ex situ. This is achieved by isolating CTCs using methods well-known in the art and contacting them with a fusogenic liposome of any one of the embodiments above.

In certain embodiments, the cancer selectively detected or treated according to any one of the above embodiments is selected from the group of breast cancer, such as triple-negative breast cancer, melanoma, lung cancer, thyroid cancer and prostate cancer.

In certain embodiments, the method intended for selectively detecting cancer cells is used for selectively detecting cancer cells and treating cancer, said method comprising systemically administering or topically applying the fusogenic liposome and optionally the functionalized detectable agent, wherein the fusogenic liposome comprises said detectable agent and a cytotoxic agent and/or immune system activating agent; in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent; and optionally monitoring treatment response by repeatedly detecting said detectable agent over time.

In another aspect, the present invention provides a method for treating cancer, comprising administering a fusogenic liposome comprising or encapsulating in its internal aqueous compartment a cytotoxic drug or a photodynamic agent, wherein said fusogenic liposome (a) comprises a lipid bilayer comprising a plurality of lipid molecules having 14 to 24 carbon atoms, wherein at least one of said lipid molecules further comprises a cationic group, a cationic natural or synthetic polymer, a cationic amino sugar, a cationic polyamino acid or an amphiphilic cancer-cell binding peptide; and at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide; (b) optionally a first functional group of a specific binding pair capable of binding to a complementary second functional group of said binding pair; and (c) in case (b) exists, optionally an immune-system activating agent comprising said complementary second functional group of said binding pair bound to said first functional group.

In another aspect, the present invention provides a fusogenic liposome for use in treating cancer, wherein the fusogenic liposome comprises or encapsulates in its internal aqueous compartment a cytotoxic drug or a photodynamic agent, wherein said fusogenic liposome (a) comprises a lipid bilayer comprising a plurality of lipid molecules having 14 to 24 carbon atoms, wherein at least one of said lipid molecules further comprises a cationic group, a cationic natural or synthetic polymer, a cationic amino sugar, a cationic polyamino acid or an amphiphilic cancer-cell binding peptide; and at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide; (b) optionally a first functional group of a specific binding pair capable of binding to a complementary second functional group of said binding pair; and (c) in case (b) exists, optionally an immune-system activating agent comprising said complementary second functional group of said binding pair bound to said first functional group.

In certain embodiments, in case (b) but not (c) exists, the method or use further comprising administering an immune-system activating agent functionalised with a complementary second functional group of the binding pair capable of binding to said first functional group of said lipid molecules.

In certain embodiments, the cytotoxic drug is a chemotherapeutic agent, such as alkylating agents (e.g., cyclophosphamide, ifosfamide, melphalan, chlorambucil, aziridines, epoxides, alkyl sulfonates), cisplatin and its analogues (e.g., carboplatin, oxaliplatin), antimetabolitites (e.g., methotrexate, 5-fluorouracil, capecitabine, cytarabine, gemcitabine, fludarabine), toposiomerase interactive agents (e.g., camptothecin, irinotecan, topotecan, etoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, interleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, anti-androgens (e.g., flutamide, casodex), kinase and tyrosine inhibitors (e.g., imatinib (STI571 or Gleevac); gefitinib (Iressa); and erlotinib (Tarceva), amphiphilic cancer-cell binding peptide is selected from Cecropin A; Cecropin A 1-8; and cyclic CNGRC; photodynamic agent such as Photofrin, Gold.

In yet another aspect, the present invention provides a kit comprising: (a) a first container comprising a fusogenic liposome comprising a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide; and wherein at least one of said lipid molecules is functionalised with a first functional group of a specific binding pair capable of binding to a complementary second functional group of said binding pair; (b) a second container comprising a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) a pamphlet with instructions for a method for selectively detecting cancer cells comprising administering to a cancer patient the fusogenic liposome of (a) and subsequently the detectable agent of (b).

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The compositions may be formulated for parenteral administration by injection, e.g., by bolus injection or continuous infusion or direct-tumor injection. Formulations for injection may be presented in unit dosage form, e.g., in ampoules or in multidose containers, with an added preservative or stabilizer. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may be formulated in liquid form/mucoadhesive formulation optionally as oral wash to deliver liposomes to mouth cancer and possibly to head and neck, esophageal cancers and other upper GI tract tumor lesions in a topical manner. The liquid form may be solutions, syrups or suspensions, or it may be presented as a drug product for reconstitution with water, injectable isotonic, or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate). The tablets may be coated by methods well-known in the art.

For buccal administration, the compositions may take the form of tablets, muco-adhesive patches/stickers or lozenges formulated in conventional manner.

The compositions may also be formulated in rectal compositions such as retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin or glycerol, for use in an inhaler or insufflator may be formulated containing a powder mix of the compound and a suitable powder base such as lactose or starch.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

While certain features of the present application have been illustrated and described herein, many modifications, substitutions, changes, and equivalents will be apparent to those of ordinary skill in the art. It is, therefore, to be understood that the appended claims are intended to cover all such modifications and changes as fall within the true spirit of the present application.

In summary, according to the embodiment 1, the present invention provides a fusogenic liposome comprising a detectable agent and optionally a cytotoxic drug in its internal aqueous compartment or bound to the liposome membrane; wherein

said fusogenic liposome comprises a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; and

at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide.

Embodiment 2: The method of claim 1, wherein said detectable agent is selected from a fluorescent probe, a contrast agent for magnetic resonance imaging (MRI), computed tomography (CT) or positron emission tomography (PET), and a photodynamic agent.

Embodiment 3: The fusogenic liposome of embodiment 1, wherein the detectable agent is a fluorescent probe selected from cy3, cy5, cy5.5, cy7 cy9, FITC, fluorescein, alexa fluor 790, alexa fluor 750, alexa fluor 700, alexa fluor 680, alexa fluor 660, alexa fluor 647, alexa fluor 633, alexa fluor 594, Qdots ranging 585 nm to 800 nm, fluorescent protoporphyrin oligomers, isocyanine green (ICG); or

an activatable fluorescent probe selected from fluorescein analogs (such as di-acetate modified analogs), coumarin analogs (such as py+BC690-(1-Methyl-4-(2-oxo-8-(pyrrolidin-1-yl)-2H-benzo[g]chromen-3-yl)pyridinium trifluoromethanesulfonate)), CFSE (5(6)-Carboxyfluorescein diacetate N-succinimidyl ester), rhodamine analogs (such as gGlu-HMRG (γ-glutamyl hydroxymethyl rhodamine green)), curcuminoid difluoroboron-based tumor-targeting γ-glutamyltranspeptidase (GGT)-activatable) fluorescent probe (Glu-DFB), and an indocyanine analog (such as AP-Glu (3H-Indolium, 2-[(1E)-2-[4-[[4-[[(4S)-4-amino-4-carboxy-1-oxobutyl]amino]phenyl]methoxy]phenyl]ethenyl]-1-(5-carboxypentyl)-3,3-dimethyl-, bromide (1:1), CAS Registry Number 1884698-06-9) and other near-infrared fluorescence activated molecule.

Embodiment 4: The fusogenic liposome of aspect 1, wherein the contrast agent for MRI is selected from iron oxide contrast agents (such as magnetite, Fe₃O₄); barium sulfate; and gadolinium contrast agents, such as gadoterate, gadodiamide, gadobenate, gadopentetate, gadobutrol; the contrast agent for CT is selected from metal elements, such as iodine, bismuth, bromine, tantalum, gold, platinum, ytterbium, yttrium, gadolinium, tungsten, indium and lutetium; or the contrast agent for PET is selected from ⁶⁴Cu-PSTM, ¹⁸F-FDG, ¹⁸F-fluoride, ¹⁸F-fluoromisonidazole and Gallium.

Embodiment 5: The fusogenic liposome of any one of embodiments 1 to 4, wherein at least one of said lipid molecules is functionalised with a first functional group of a specific binding pair capable of binding to a complementary second functional group of said binding pair.

Embodiment 6: The fusogenic liposome of embodiment 5, wherein the fusogenic liposome further comprises a first spacer between the lipid bilayer and the first functional group.

Embodiment 7: The fusogenic liposome of embodiments 5 or 6, further comprising an identical or different additional detectable agent or an immune system activating agent, each one functionalised with said complementary second functional group and bound to said first functional group via said second functional group, wherein said identical or different additional detectable agent is selected from a fluorescent probe and a contrast agent for magnetic resonance imaging (MRI), computed tomography (CT) or positron emission tomography (PET).

Embodiment 8: The fusogenic liposome of embodiment 7, wherein said immune system activating agent is agent is selected from anti-CD3 antibody, an anti-CD8 antibody, an anti-NKG2D antibody, or a combination thereof, an antibody capable of binding both CD3 and CD8 and an antibody capable of binding both CD3 and NKG2D.

Embodiment 9: The fusogenic liposome of any one of embodiments 5 to 8, wherein said detectable agent or immune system activating agent is bound at the outer leaflet of the fusogenic liposome.

Embodiment 10: The fusogenic liposome of any one of embodiments 5 to 9, wherein the detectable agent or immune-system activating agent further comprises a second spacer between the detectable agent or immune-system activating agent and the second functional group.

Embodiment 11: The fusogenic liposome of embodiment 10, wherein the first or second spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide, such as a poly-Gly peptide of about 2-20 amino acid residues in length, e.g. 3 amino acid residues in length.

Embodiment 12: The fusogenic liposome of embodiment 11, wherein the first or second spacer is PEG of molecular weight of about 106 Da to about 4 kDa.

Embodiment 13: The fusogenic liposome of embodiment 12, wherein PEG is of a molecular weight of about 194 Da (PEG₄).

Embodiment 14: The fusogenic liposome of embodiment 10, wherein the first or second spacer is (C₆-C₁₂)alkyl, preferably heptyl or dodecanoyl.

Embodiment 15: The fusogenic liposome of any one of embodiments 1 to 14, wherein said at least one of said lipid molecules comprising a cationic group is selected from 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), Dimethyldioctadecylammonium (18:0 DDAB), and N1-[2-((1S)-1-[(3-aminopropyl)amino]-4-[di(3-amino-propyl)amino]butyl-carboxamido)ethyl]-3,4-di[oleyloxy]-benzamide (MVL5).

Embodiment 16: The fusogenic liposome of embodiment 15, wherein said at least one of said lipid molecules comprising a cationic group is DOTAP.

Embodiment 17: The fusogenic liposome of any one of embodiments 1 to 14, wherein said cationic synthetic polymer is selected from polyethyleneimines (PEI) and poly(2-(dimethylamino)ethyl methacrylate.

Embodiment 18: The fusogenic liposome of any one of embodiments 1 to 14, wherein said cationic natural polymer is chitosan.

Embodiment 19: The fusogenic liposome of any one of embodiments 1 to 14, wherein said cationic amino sugar is glucosamine.

Embodiment 20: The fusogenic liposome of any one of embodiments 1 to 14, wherein said cationic polyamino acid is selected from poly(L-lysine), poly(L-arginine), poly(D-lysine), poly(D-arginine), poly(L-ornithine) and poly(D-ornithine).

Embodiment 21: The fusogenic liposome of any one of embodiments 1 to 14, wherein said amphiphilic cancer-cell binding peptide is selected from Cecropin A; Cecropin A 1-8; and cyclic CNGRC.

Embodiment 22: The fusogenic liposome of any one of embodiments 1 to 21, wherein said at least one of said lipid molecules is a phospholipid selected from the group consisting of a phosphatidylcholine, a phosphatidylethanolamine, a phosphatidylserine, a phosphatidic acid or a combination thereof, each one of which comprises one or two identical or different fatty acid residues, wherein the fatty acid residues in the phosphatidyl moiety is saturated, mono-unsaturated or poly-unsaturated and has a carbon chain length of 14, 15, 16, 17, 18, 19, 20, 21, 22, 23 or 24 carbons, such as myristoyl, stearoyl, palmitoyl, oleoyl, linoleoyl, linolenoyl (including conjugated linolenoyl), arachidonoyl in phospholipid and lyso-phospholipid configuration, and combinations thereof.

Embodiment 23: The fusogenic liposome of embodiment 22, wherein said phospholipid is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-distearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (Δ9-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-distearoyl-3-phosphatidylethanolamine (DSPE); 1,2-dimyristoyl-3-phosphatidylserine (DMPS); 1,2-dipalmitoyl-3-phosphatidylserine (DPPS); palmitoyloleoyl phosphatidylethanolamine (POPE); and 1,2-dioleoyl-3-phosphatidylserine (DOPS).

Embodiment 24: The fusogenic liposome of embodiment 23, wherein said phospholipid is selected from DOPC, POPC, DMPC, DPPC, DOPE, POPE, DSPE, DMPE and DPPE.

Embodiment 25: The fusogenic liposome of any one of embodiments 1 to 24, wherein the stabilizing moiety is PEG of molecular weight of about 106 Da to about 4 kDa.

Embodiment 26: The fusogenic liposome of embodiment 25, wherein PEG is of molecular weight of about 2 kDa.

Embodiment 27: The fusogenic liposome of any one of embodiments 1 to 26, wherein said stabilizing moiety is connected to at least one of said lipid molecules via a cleavable peptide linker.

Embodiment 28: The fusogenic liposome of any one of embodiments 4 to 27, wherein said first functional group of the specific binding pair is capable of forming a covalent bond with said complementary second functional group of said binding pair.

Embodiment 29: The fusogenic liposome of embodiment 28, wherein said first functional group of the specific binding pair is capable of forming a covalent bond with said complementary second functional group of said binding pair via a click chemistry reaction.

Embodiment 30: The fusogenic liposome of embodiment 28, wherein i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa; iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or derivative thereof such as ester, anhydride, acyl halide, tosyl and N-hydrosuccinimide (NHS), and the second functional group of said binding pair is amine, or vice versa; viii) functional group.

Embodiment 31: The fusogenic liposome of embodiment 30, wherein the specific binding pair is alkyne-azide.

Embodiment 32: The fusogenic liposome of any one of embodiments 4 to 27, wherein said first functional group of the specific binding pair is capable of forming a non-covalent bond with said complementary second functional group of said binding pair.

Embodiment 33: The fusogenic liposome of embodiment 32, wherein the first functional group of the specific binding pair is biotin, and the second functional group of said binding pair is its binding-partner selected from a biotin-binding peptide or biotin-binding protein, or vice versa.

Embodiment 34: The fusogenic liposome of embodiment 33, wherein said biotin-binding protein is selected from avidin, streptavidin and an anti-biotin antibody.

Embodiment 35: The fusogenic liposome of embodiment 34, wherein said biotin-binding peptide is selected from AEGEFCSWAPPKASCGDPAK (SEQ ID NO: 1), CSWRPPFRAVC (SEQ ID NO: 2), CSWAPPFKASC (SEQ ID NO: 3), and CNWTPPFKTRC (SEQ ID NO: 4).

Embodiment 36: The fusogenic liposome of any one of embodiments 1 to 35, wherein the fusogenic liposome further comprises cholesterol (CHO) or its derivatives.

Embodiment 37: The fusogenic liposome of any one of embodiments 1 to 36, wherein the fusogenic liposome comprises DOPC:DOTAP:DSPE-PEG2K:DOPE or DOPC:DOTAP:DSPE-PEG2K, and optionally cholesterol, wherein PEG2K represents PEG having a molecular weight of about 2 kDa, and the relative molar amount of DOPC is up to about 80%, the relative molar amount of DOTAP is up to about 80%, the relative molar amount of DSPE-PEG2K is up to about 20%, the relative molar amount of DOPE is up to about 20%, the relative molar amount of cholesterol is up to about 40%.

Embodiment 38: The fusogenic liposome of embodiment 37, wherein the fusogenic liposome comprises:
DOPC:DOTAP:DSPE-PEG2K:DOPE in the molar ratio 52.5:35:0.6:10, 52.5:35:1.25:10, 52.5:35:2.5:10, 52.5:35:5:10, 52.5:35:0.6:5, 52.5:35:1.25:5, 52.5:35:2.5:5, 52.5:35:5:5, 65:20:5:10, 50:35:5:10, 52.5:35:1.25:7, 52.5:35:1.25:5, or 52.5:35:2.5:7; or
DOPC:DOTAP:DSPE-PEG2K, in the molar ratio 52.5:35:0.6, 52.5:35:1.25, 52.5:35:2.5, 52.5:35:5, 52.5:35:0.6, 52.5:35:1.25, 52.5:35:2.5, 52.5:35:5, 65:20:5, 50:35:5, 52.5:35:1.25, 52.5:35:1.25, or 52.5:35:2.5.

Embodiment 39: The fusogenic liposome of embodiment 38, wherein the fusogenic liposome comprises DOPC:DOTAP:DSPE-PEG2K:DOPE in the molar ratio 52.5:35:2.5:5; or DOPC:DOTAP:DSPE-PEG2K, in the molar ratio 52.5:35:2.5.

Embodiment 40: The fusogenic liposome of any one of embodiments 1 to 39, wherein the melting temperature (Tm) of the liposome is below 45° C., at which the fusogenic liposome is maintained at a non-crystalline transition phase thereby providing membrane fluidity required for fusion of liposome with cell membranes.

Embodiment 41: The fusogenic liposome of any one of embodiments 1 to 40, wherein the fusogenic liposome has a size of up to 200 nm, e.g. from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, e.g. about 100 nm.

Embodiment 42: A method for selectively detecting cancer cells, comprising contacting said cancer cells with a fusogenic liposome of any one of embodiments 1 to 39; and in case said detectable agent is an activatable fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent.

Embodiment 43: The method of embodiment 42, wherein selective detection of cancer cells in a cancer patient indicates responsiveness of said cancer patient to treatment of cancer with a cancer drug comprised in a nanoparticle.

Embodiment 44: The method of embodiment 43, wherein the cancer patient determined as responsive is treated with said nanoparticle comprising said cancer drug.

Embodiment 45: A method for selectively detecting cancer cells, comprising (a) contacting said cancer cells with a functionalised fusogenic liposome of any one of embodiments 4 to 41; (b) contacting said cancer cells with a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) in case said detectable agent is a fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby selectively detecting said cancer cells.

Embodiment 46: The method of any one of embodiments 42 to 45, wherein said detectable agent is (a) a fluorescent probe selected from cy3, cy5, cy5.5, cy7 cy9, FITC, fluorescein, alexa fluor 790, alexa fluor 750, alexa fluor 700, alexa fluor 680, alexa fluor 660, alexa fluor 647, alexa fluor 633, alexa fluor 594, Qdots ranging 585 nm to 800 nm, fluorescent protoporphyrin oligomers, isocyanine green (ICG); or (b) an activatable fluorescent probe selected from fluorescein analogs (such as di-acetate modified analogs), coumarin analogs (such as py+BC690-(1-Methyl-4-(2-oxo-8-(pyrrolidin-1-yl)-2H-benzo[g]chromen-3-yl)pyridinium trifluoromethanesulfonate)), CFSE (5(6)-Carboxyfluorescein diacetate N-succinimidyl ester), rhodamine analogs (such as gGlu-HMRG (γ-glutamyl hydroxymethyl rhodamine green)), curcuminoid difluoroboron-based tumor-targeting γ-glutamyltranspeptidase (GGT)-activatable) fluorescent probe (Glu-DFB), and an indocyanine analog, such as AP-Glu (3H-Indolium, 2-[(1E)-2-[4-[[4-[[(4S)-4-amino-4-carboxy-1-oxobutyl]amino]phenyl]methoxy]phenyl]ethenyl]-1-(5-carboxypentyl)-3,3-dimethyl-, bromide (1:1) and other near-infrared fluorescence activated molecule. The ester modification(s) on the activatable fluorophore render it non-fluorescent, and it becomes fluorescent upon fusion of the liposome with a cancer cell and subsequent cytoplasmic cleavage by cytoplasmic esterases.

Embodiment 47: The method of embodiment 45 or 46, wherein selective detection of cancer cells in a cancer patient indicates responsiveness of said cancer patient to treatment of cancer with a cancer drug comprised in a nanoparticle.

Embodiment 48: The method of embodiment 47, wherein the cancer patient determined as responsive is treated with said nanoparticle comprising said cancer drug.

Embodiment 49: A method for treating cancer by fluorescence-guided surgery or targeted radiotherapy, said method comprising the method of any one of embodiments 42 to 48 and removing the tumor containing the cancer cells.

Embodiment 50: The method of any one of embodiments 42 to 48, wherein said cancer patient is undergoing imaging of tumors, such as skin cancer, or whole body imaging, said method comprising systemically administering or topically applying the fusogenic liposome, and optionally the functionalized detectable agent; and, in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, scanning an area of an organ, such as skin, or whole body, thereby defining tumor location and margins.

Embodiment 51: A method for treating cancer comprising the method of embodiment 50 and removing the tumor containing the cancer cells.

Embodiment 52: The method of embodiment 42 or 45, for selectively detecting cancer cells ex situ in a tissue or a blood-derived fraction removed from the cancer patient in need thereof, said method comprising systemically administering or applying the fusogenic liposome, and optionally the functionalized fluorescent probe, to the tissue or blood-derived fraction and detecting said fluorescent probe by illuminating the tissue or blood-derived fraction and detecting light emitted from the fluorescent probe, thereby selectively detecting said cancer cells.

Embodiment 53: The method of embodiment 52, wherein said tissue is skin, said cancer patient is undergoing surgery for removal of skin cancer (Mohs surgery), and said surgery is repeated until the skin tissue has no detectable cancer cells.

Embodiment 54: The method of embodiment 52, for selectively detecting circulating tumour cells (CTCs) in said blood-derived fraction.

Embodiment 55: The method of any one of embodiments 42 to 54, wherein said cancer is selected from the group of breast cancer, such as triple-negative breast cancer, melanoma, lung cancer, thyroid cancer and prostate cancer.

Embodiment 56: The method of embodiment 42 or 45, for selectively detecting cancer cells and treating cancer, said method comprising systemically administering or topically applying the fusogenic liposome and optionally the functionalized detectable agent, wherein the fusogenic liposome comprises said detectable agent and a cytotoxic agent and/or immune system activating agent; in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent; and optionally monitoring treatment response by repeatedly detecting said detectable agent over time.

Embodiment 57: A kit comprising: (a) a first container comprising a fusogenic liposome of any one of embodiments 5 to 41; (b) a second container comprising a detectable agent selected from a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and (c) a pamphlet with instructions for a method for selectively detecting cancer cells comprising administering to a cancer patient the fusogenic liposome of (a) and subsequently the detectable agent of (b).

The invention will now be illustrated by the following non-limiting examples.

### EXAMPLES

### Materials and Methods:

Lipids: DOPE (1,2-dioleoyl-sn-glycero-3-phosphoethanolamine, Lipoid), DSPE (1,2-Distearoyl-sn-glycero-3-phosphoethanolamine), DOPC (1,2-Dioleoyl-sn-glycero-3-phosphocholine, DOPC, Lipoid), HSPC(Hydrogenised soy phosphocholine, Lipoid), DOTAP (1,2-Dioleoyloxy-3-Trimethylammoniumpropanchloride, Lipoid), DSPE-PEG2000 (N-(Carbonyl-methoxypolyethylenglycol-2000)-1,2-distearoyl-sn-glycero-3-phosphoethanolamine, MPEG-2000-DSPE, Lipoid), Cholesterol (Sigma), DOPE-FITC (in house synthesis), DSPE-PEG4-Azide (in house synthesis), DOPE-PEG4-Azide (in house synthesis), DSPE-PEG4-Biotin (in house synthesis).

PEG4 represents PEG having a molecular weight of about 194 Da.

Isocyanine green, Cardiogreen (Sigma, cat number 21980).

Ethanol absolute is used to dissolve lipids at 70° C.

### Production of Detectable Liposomes Using Thin Film Hydration:

Dissolve lipids in chloroform or chloroform methanol mixtures to enable full solubilization in glass bulb. Evaporate under vacuum or under nitrogen stream while constantly rotating bulb to enable uniform film formation. Let solvents evaporate for 1 hour under vacuum after visible solvents have evaporated.

Pre-warm aqueous buffer (such as PBS) with detectible agent such as Gd-DTPA or ICG above the highest Tm according to phospholipids used. Heat bulb to same temperature and add aqueous buffer to lipid film. Vortex to mix and rotate for 60 minutes. Downsize into liposomes using extruder or probe sonication or microfluidics-based flow cells. Remove un-encapsulated material using dialysis or size exclusion methods. Modify ethanol-amine group using NHS-linker-second functional group at 5 molar excess for 1 hr at 25° C., mixing at 400 RPM. Remove unbound linker-second functional group using dialysis or size exclusion methods.

### Production of Immune Labelling Liposomes Using Ethanol Injection:

Liposome preparation is a technique well-known in the art and can be performed for example according to protocols disclosed in Torchilin V. P. and Weissig V., Liposomes: a practical approach 2^{nd} edition (OUP Oxford, 2003).

In our case, lipids (Avanti-polar lipids or lipoid) were weighed according to the required composition and were solubilized in EtOH absolute at final volume of 10% of the required liposome volume. Lipids-EtOH mixture was heated above the Tm (melting temperature) of the lipids. EtOH injection was performed into PBS without calcium or magnesium, pH=7.2-7.4 at identical temperature and the lipid-buffer was mixed and extruded to yield liposomes at the desired size distribution of about 90 nm to 100 nm using an extruder.

Alternatively, lipids were solubilized in chloroform or chloroform-MeOH (2:1) or chloroform-MeOH (3:1) in a glass container and mixed thoroughly to achieve homogeneous solution. Lipids in organic solvents were then evaporated under negative pressure (150 mBar) for 2 hrs to remove the organic solvent thereby creating a thin film of lipids on the glass container. The lipid film was hydrated using a pre-heated (above lipid Tm) and concentrated aqueous solution containing a fluorophore (such as PBS/0.9% NaCl/DDW/5% dextrose, the content of which depends on the fluorophore of interest. For example, indocyanine green (ICG, Cardiogreen) is readily solubilized in 5% dextrose and enables liposome encapsulation. However, ICG creates aggregates in phosphate buffered saline (PBS)—and is therefore incompatible with PBS for purpose of liposome encapsulation.

If the fluorophore is linked to a lipid (such as fluorescein isothiocyanate (FITC)-DOPE, the fluorophore is added to the lipid mixture and dissolved in EtOH prior to EtOH injection. Such a fluorescently labeled lipid can also be used in thin film hydration method or using nano-assembler or other microfluidics-based liposome assembly methods. The lipid-aqueous solution was then mixed (vortex) and heated above Tm. If precipitates were visible, sonication was performed until film is fully hydrated and a uniform milky solution is achieved.

A non-limiting example named N8 formulation was typically used to selectively fuse with cancer cells in the studies shown below:
DOTAP:DOPC:DOPE:DSPE-PEG2000 at molar ratio of 35:52.5:10:2.5

Liposome size determination is routinely done, and was done in the present case, using dynamic light scattering, (DLS, Malvern instruments) and can also be determined using cryo-TEM electron microscopy.

Tm determination: Tm is determined by using Differential Scanning Calorimetry (DSC), a machine that heats up and then cools down the sample and measures the changes in the sample. See e.g. Epand R. M. High sensitivity differential scanning calorimetry of the bilayer to hexagonal phase transitions of diacylphosphatidylethanolamines (Chemistry and Physics of Lipids 1985). We have also used the Avanti®-Polar Lipids website and Lipoid website to determine Tm.

### Fluorophore Encapsulation for Cell-Mediated Labelling:

For thin film and for ethanol injection the fluorophore (e.g. Indocyanine green (ICG)) used at 1 mg/ml concentration but can be lowered or elevated as necessary or CFSE (5(6)-Carboxyfluorescein diacetate N-succinimidyl ester) a functionalized activatable fluorophore, which can covalently bind primary amines, and has two ester modifications on the fluorescein which render it non fluorescent. It becomes fluorescent upon fusion of the liposome with a cancer cell and subsequent cytoplasmic cleavage. Other molecules with similar ester modifications are available by ThermoFisher (cell trace: Blue, Violet, CFSE, Yellow, Far Red and can be used at excitation/emission wavelengths of 375/410 nm, 405/450 nm, 495/519 nm, 546/579 nm, 630/661 nm, respectively).

Excess of non-encapsulated fluorophore was removed using dialysis (5-1 ml liposomes against 2 L buffer, replaced at least 3 times over 24 hrs) or using size exclusion methods. When using ethanolamine lipids such as DOPE, the NHS in CFSE (and other similar Thermofisher molecular probes) binds covalently to the lipid which improves stability of the fluorescent liposome, since the fluorophore cannot leak out of the liposome.

When preparing liposomes with activatable fluorophores, encapsulation of these fluorophores must be done under conditions that do not cleave the ester bound group. Basic pH conditions and elevated temperatures result in methyl-Esther cleavage from fluorescein and will result in false positive.

### Liposome Modification Post Production:

Liposomes containing ethanolamine groups were chemically modified post extrusion with a linker and azide (one member of a binding pair) using the NHS ester chemical reaction (N-hydroxysuccinimide). Typically, the NHS-polyethylene glycol (PEG)4-Azide (NHS group) is used at 5 molar equivalents per primary amine group (DOPE lipid). The unbound excess was removed using size exclusion chromatography.

Liposomes were alternatively made using a pre-modified lipid to yield a similar liposomal product that allows a copper dependent or independent click reaction. Briefly, a DSPE or DOPE lipid pre-modified with PEG4-alkyne or azide was incorporated into lipid mixture prior to EtOH injection.

### Creating 2STEP and OUT Approaches Using Modified Antibodies and Modified Liposomes:

2STEP: In this approach, cancel cells are labelled in two steps. First, the cells are contacted with functionalized liposomes, and in a second step the cells are contacted with a functionalized antibody or detectable probe or nano-particles for MRI/CT/PET/fluorescence. Liposomes covalently linked to one member of the binding pair (e.g. azide), were used directly on cells at the appropriate dilution (or injected intravenously (IV) in animal models) followed by washes of treated cells (not applicable under in vivo settings) and were allowed to react with antibodies, a fluorescent probe (Indocyanine green), or nano-particles for MRI/CT/PET/fluorescence modified with the complementary member of the binding pair (e.g. BCN).

OUT: In this approach, cancer cells are labelled in one step with liposomes carrying a functionalized antibody or detectable probe on their outer leaflet. Liposomes covalently linked to one member of the binding pair (e.g. azide), were allowed to react with antibodies or Indocyanine green with the complementary member of the binding pair (e.g. BCN). The modified liposomes were then used directly on cells at the appropriate dilution (or injected IV under animal models) followed by washes of treated cells (not applicable under in-vivo settings)

### Example 1: In Situ Detection of Triple Negative Breast Cancer Using One-Step Procedure

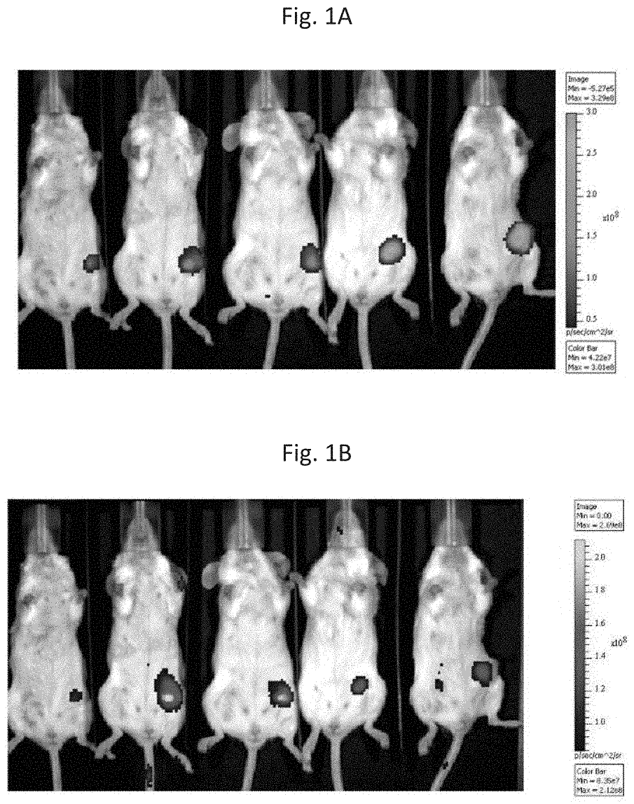

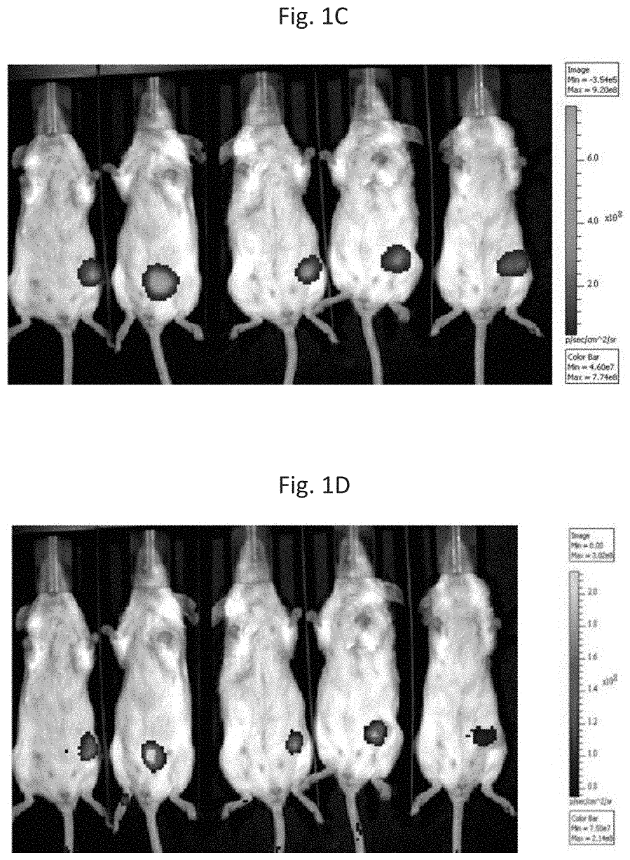

Indocyanine green (ICG) containing liposomes were injected IV into 4T1mCherry (triple negative breast cancer) tumor bearing mice. 24 hrs post liposome injection, the mice were imaged for ICG signal and for mCherry using IVIS® Spectrum in vivo imaging system (PerkinElmer). Images on the left panel show tumor mCherry signal (FIGS. 1A and C) and images on the right panel (FIGS. 1B and D) show ICG signal recorded 24 hrs post injection of ICG loaded liposomes modified with PEG₄-Azide (C) or with PEG₄-Biotin (D) as examples of the first binding pairs. The presence of the azide or biotin functional groups enables a theranostic use of the liposomes by administration of T cell activating agents, such as BCN- or avidin-functionalized anti-CD3 and anti-CD8 antibodies, that will bind to the labeled cancer cells and thus this method not only visualizes the cancer cells but also evokes a T cell response against them.

Fluorescent signal shows co-localization of our liposomes into the fluorescent tumors as seen in FIGS. 1A-D, where the liposome signal (ICG) is overlapping with the mCherry signal.

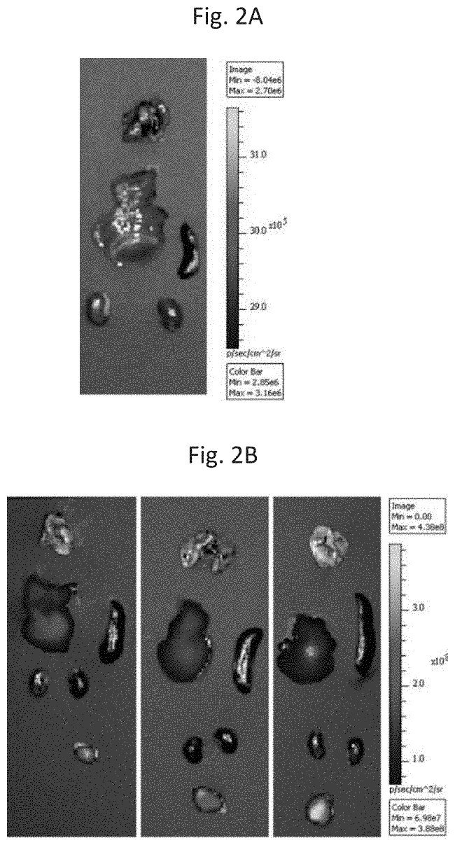

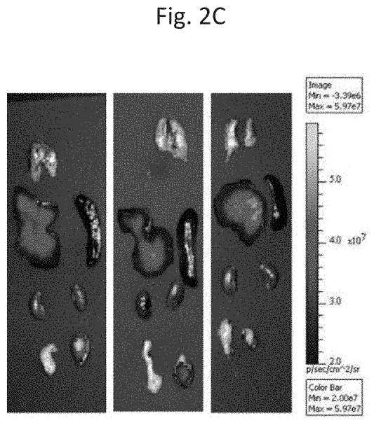

Data shown in FIGS. 2A-C, although gathered post mortem, show that the fusogenic liposomes can be used in fluorescent imaging assisted surgical procedures, photodynamic therapy (assuming relevant photodynamic fluorophore is used) or irradiation of cancerous mass, and for analyzing biopsies ex situ.

Similarly, MRI/CT/PET contrast agent containing liposomes are injected IV into 4T1mCherry (triple negative breast cancer) tumor bearing mice. 24 hrs post liposome injection, the mice are imaged for mCherry using IVIS® Spectrum in vivo imaging system (PerkinElmer) and for the MRI/CT/PET contrast agent signal using appropriate imaging device.

### Example 2. In Situ Detection of Cancer Using Two-Step Procedure

Fusogenic liposomes comprising a first functional group of a specific binding pair, such as azide, are injected intravenously and 30 minutes to 48 Hrs later a second nano-particle comprising the complementary functional group carrying a detectable probe (such as BCN-functionalized nano-gold particles or BCN-iodine carrying liposomes that are detectable in CT or BCN-bound ICG loaded liposomes) is injected. The circulation half-life time and pharmacodynamic profiles of the first fusogenic liposome and the second nano-particle dictates the optimal time to imaging and can be used to avoid off target labeling in organs such as liver and spleen.

The use of two types of liposomes, or two nano-technology based particles or combinations thereof can improve signal to noise and reduce false positives. One liposome with a first binding pair, that selectively fuse with cancer cells, and the second nanoparticle, with the second binding pair, to help detect the fused cells can be also used. This combination (positively charged liposome with other CT/MRI/fluorescent detectable liposome with longer circulation time or with gold nano-particle or other polymeric nano-particles) can be used in a two-step injection manner to improve signal to noise and reduce false positive in tissues such as liver and spleen. The first liposome undergoes selective fusion with cancer cells, adding first binding pair to the cancer cells' membrane outer leaflet, whereas the second nano-particle binds using the second binding pair. By injection liposomes first and at least two half-life times apart, injecting the second nano-particle we can improve signal to noise in liver and spleen. Fluorescence resonance energy transfer (FRET) can also be used under in-vivo conditions, where the first fusogenic liposome carries one fluorophore bound to lipid and the first functional group of a specific binding pair, and the second liposome/nanoparticle carries the complementary functional group and a second fluorophore that can be used in FRET and will yield signal if two liposomes with the two fluorophores come in close contact, meaning when membrane of liposome labelled cancer cells is close to the second liposome with the other fluorophore, FRET is possible and signal fluorescence shift occurs.

### Example 3. In Situ Detection of Cancer Using Activatable Fluorescent Probe

Liposomes carrying an esterase cleavable fluorophore used during surgery is injected intravenously or used to irrigate tumor tissue and surrounding suspected as neoplastic tissues. The tissue is then washed with saline solution to remove unbound liposome and blood to improve signal to noise. Fusion of liposomes with cancer cells results in removal of the ester groups on the fluorophore and allows detection using fluorescent excitation and emission wavelengths corresponding with the fluorophore used.

This technique is then utilized in fluorescence-guided surgery or targeted radiotherapy to remove the visualized tumors.

### Example 4. Selectively Detecting Cancer Cells Ex Situ

One-step procedure: A tissue suspected of neoplasia, such as skin in the case of Mohs surgery, is taken out and washed with PBS or other buffer to remove blood clots, to improve signal to noise and is immersed for 15 minutes in activatable fluorophore liposomes solution at 5 mM lipids. Liposome-treated tissue is washed with PBS or other physiological isotonic solution (such as saline) and is imaged under fluorescent microscope with excitation and emission wavelengths corresponding with fluorophore.

Two step procedure: A tissue suspected of neoplasia is taken out and washed with PBS or other buffer to remove blood clots, to improve signal to noise and is immersed for 15 minutes in selective fusogenic liposomes comprising a functional group of a specific binding pair, such as azide (e.g. at 5 mM lipids). Liposome treated-tissue is washed with PBS or other physiological isotonic solution (such as saline) and is immersed in 4° C. solution of 5-500 micrograms per ml of a fluorophore comprising the complementary functional group, such as DBCO-Cy5 for 1 hr, washed and imaged under fluorescent microscope with excitation and emission wavelengths corresponding with fluorophore.

If cancer cells are detected in the analyzed tissue sample, additional tissue is removed from the suspected area and analyzed as described above until no signal is detected in the sample.

### Example 5. Detection of Circulating Cancer Cells (CTCs) in Blood

Different devices are used for isolation of circulating live tumor cells, such as filtration.

These cells are co-isolated with leukocytes that require anti-CD45 staining for identification. Cancer cells do not always have a known target marker, and therefore, selective fusogenic liposomes carrying a fluorescent dye or an activatable dye can be used to directly label cancer cells isolated from blood.

### Example 6. Systemically Treating Tumors, Detecting Tumor Presence and Predicting Treatment Efficacy

Tumor bearing mice were administered with Gd and ICP co-encapsulated liposomes, bound to anti-CD3 and anti-CD8 (effector T cell activating liposomes) at a single dose or at repeated doses where factor tested is time between injections. These liposomes can be integrated with DSPE-DTPA(Gd), 1,2-distearoyl-sn-glycero-3-phosphoethanolamine-N-diethylenetriaminepentaacetic acid (gadolinium salt) that has the DTPA chelator pre-bound to lipid head group (Avanti polar lipids).

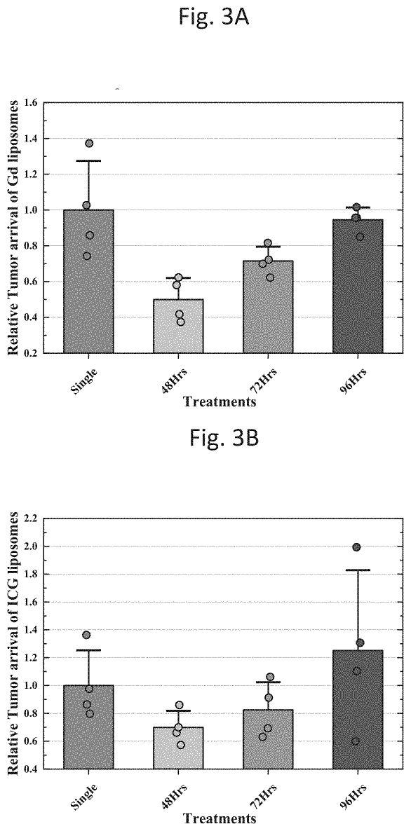

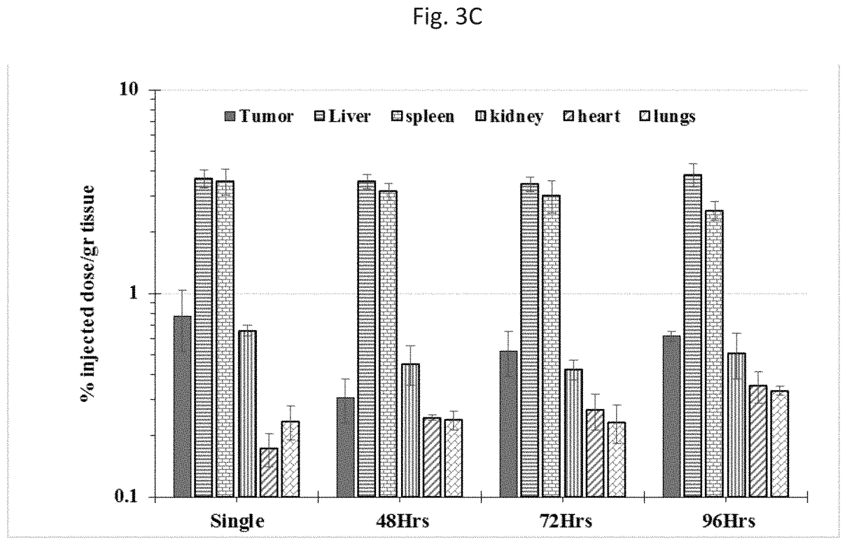

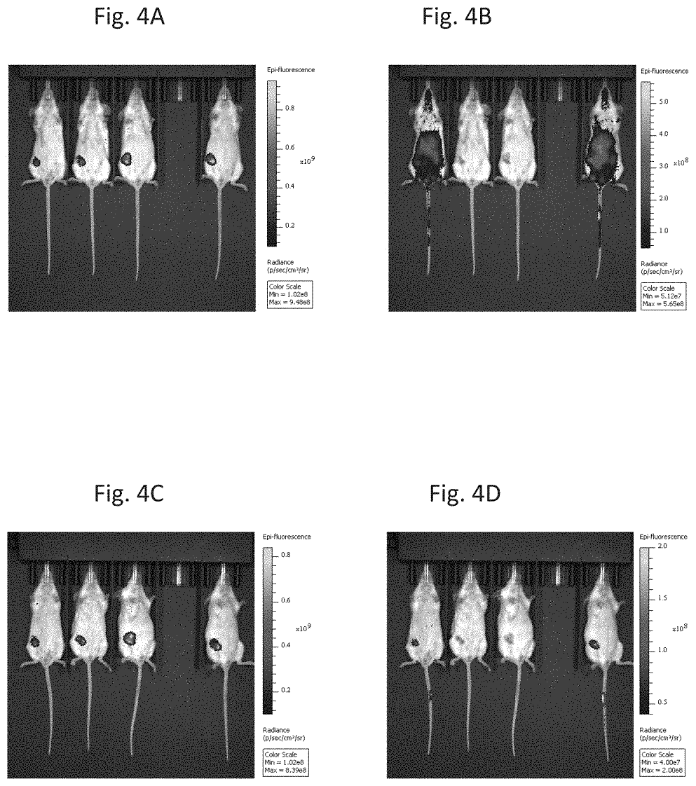

Second treatment was given to mice whose first treatment was empty liposomes with anti-CD3 and anti-CD8 antibodies at identical dose (100 microgram each mAb per mouse). Second treatment interval tested was 48 Hrs apart, 48 Hrs apart, and 96 Hrs apart. Tumor fluorophore arrival and accumulation at 24 Hrs post second treatment is presented in FIGS. 3A-C using Gd and ICG measurements. Gd signal collected from tumors post mortem and ICG signal recorded during in-vivo study (living mice) correlates well and shows similar trends of arrival of second treatments to tumors. In FIG. 3C the biodistribution of the Gd loaded liposomes is presented and shows similar profiles for single and second treatments. Accumulation in liver and spleen is attributed to uptake by tissue specific macrophages/monocytes. Whole-body images are presented in FIG. 4A-D, and show clearance from entire body and accumulation in tumor site by measuring ICG signal (FIGS. 4B and D). Tumor mCherry signal is presented in FIGS. 4A and C. Quantification of ICG signal in FIG. 3B was done based on region of interest determined by mCherry tumor signal.

After acquiring images (FIGS. 4C and D), mice were sacrificed, and organs were isolated, weighed, bunt to ashes and dissolved in 1% nitric acid and filtered using 0.45 micron filters. Organ associated Gd levels were determined using ICP elemental analysis to determine percent of injected dose per gram tissue arrival (FIG. 3C).

1. A fusogenic liposome comprising a detectable agent and optionally a cytotoxic drug in its internal aqueous compartment or bound to the liposome membrane, wherein
said fusogenic liposome comprises a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide; and
at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide.

2. The fusogenic liposome of claim 1, wherein said detectable agent is selected from the group consisting of a fluorescent probe, a contrast agent for magnetic resonance imaging (MRI), computed tomography (CT) or positron emission tomography (PET), and a photodynamic agent.

3. The fusogenic liposome of claim 2, wherein said detectable agent is (a) a fluorescent probe selected from the group consisting of cy3, cy5, cy5.5, cy7 cy9, FITC, fluorescein, alexa fluor 790, alexa fluor 750, alexa fluor 700, alexa fluor 680, alexa fluor 660, alexa fluor 647, alexa fluor 633, alexa fluor 594, Qdots ranging 585 nm to 800 nm, fluorescent protoporphyrin oligomers, and isocyanine green (ICG); or (b) an activatable fluorescent probe selected from the group consisting of fluorescein-analogs, coumarin analogs, CFSE (5(6)-Carboxyfluorescein diacetate N-succinimidyl ester), rhodamine analogs, curcuminoid difluoroboron-based tumor-targeting γ-glutamyltranspeptidase (GGT)-activatable) fluorescent probe (Glu-DFB), and an indocyanine analog.

4. The fusogenic liposome of claim 2, wherein the contrast agent for MRI is selected from iron oxide contrast agents; barium sulfate; and gadolinium contrast agents; the contrast agent for CT is selected from metal elements, such as iodine, bismuth, bromine, tantalum, gold, platinum, ytterbium, yttrium, gadolinium, tungsten, indium, and lutetium; or the contrast agent for PET is selected from the group consisting of ⁶⁴Cu-PSTM, ¹⁸F-FDG, ¹⁸F-fluoride, ¹⁸F-fluoromisonidazole and Gallium.

5. The fusogenic liposome of claim 1, wherein at least one of said lipid molecules is functionalised with a first functional group of a specific binding pair capable of binding to a complementary second functional group of said binding pair.

6. The fusogenic liposome of claim 5, wherein the fusogenic liposome further comprises a first spacer between the lipid bilayer and the first functional group.

7. The fusogenic liposome of claim 5, further comprising an additional identical or different detectable agent or an immune system activating agent, each one functionalised with said complementary second functional group and bound to said first functional group via said second functional group,
wherein said additional identical or different detectable agent is selected from a fluorescent probe and a contrast agent for magnetic resonance imaging (MRI), computed tomography (CT) or positron emission tomography (PET).

8. The fusogenic liposome of claim 7, wherein said immune system activating agent is selected from the group consisting of anti-CD3 antibody, an anti-CD8 antibody, an anti-NKG2D antibody, or a combination thereof, an antibody capable of binding both CD3 and CD8 and an antibody capable of binding both CD3 and NKG2D.

9. The fusogenic liposome of claim 7, wherein said detectable agent or immune system activating agent is bound at the outer leaflet of the fusogenic liposome.

10. The fusogenic liposome of claim 7, wherein the detectable agent or immune-system activating agent further comprises a second spacer between the detectable agent or immune-system activating agent and the second functional group.

11. The fusogenic liposome of claim 6, wherein the first spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide.

12. The fusogenic liposome of claim 11, wherein the first spacer is PEG of molecular weight of about 106 Da to about 4 kDa.

13. The fusogenic liposome of claim 12, wherein PEG is of a molecular weight of about 194 Da (PEG4).

14. The fusogenic liposome of claim 11, wherein the first spacer is (C6-C12)alkyl, preferably heptyl or dodecanoyl.

15. The fusogenic liposome of claim 1, wherein said at least one of said lipid molecules comprising a cationic group is selected from the group consisting of 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), Dimethyldioctadecylammonium (18:0 DDAB), and N1-[2-((1S)-1-[(3-aminopropyl)amino]-4-[di(3-amino-propyl)amino]butyl-carboxamido)ethyl]-3,4-di[oleyloxy]-benzamide (MVL5).

16. The fusogenic liposome of claim 15, wherein said at least one of said lipid molecules comprising a cationic group is DOTAP.

17. The fusogenic liposome of claim 1, wherein said cationic synthetic polymer is selected from the group consisting of polyethyleneimines (PEI) and poly(2-(dimethylamino)ethyl methacrylate.

18. The fusogenic liposome of claim 1, wherein said cationic natural polymer is chitosan.

19. The fusogenic liposome of claim 1, wherein said cationic amino sugar is glucosamine.

20. The fusogenic liposome of claim 1, wherein said cationic polyamino acid is selected from the group consisting of poly(L-lysine), poly(L-arginine), poly(D-lysine), poly(D-arginine), poly(L-ornithine) and poly(D-ornithine).

21. The fusogenic liposome claim 1, wherein said amphiphilic cancer-cell binding peptide is selected from the group consisting of Cecropin A; Cecropin A 1-8; and cyclic CNGRC.

22. The fusogenic liposome claim 1, wherein said at least one of said lipid molecules is a phospholipid selected from the group consisting of a phosphatidylcholine, a phosphatidylethanolamine, a phosphatidylserine, a phosphatidic acid or a combination thereof, each one of which comprises one or two identical or different fatty acid residues, wherein the fatty acid residues in the phosphatidyl moiety is saturated, mono-unsaturated or poly-unsaturated and has a carbon chain length of 14, 15, 16, 17, 18, 19, 20, 21, 22, 23 or 24 carbons.

23. The fusogenic liposome of claim 22, wherein said phospholipid is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-distearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (Δ9-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-distearoyl-3-phosphatidylethanolamine (DSPE); 1,2-dimyristoyl-3-phosphatidylserine (DMPS); 1,2-dipalmitoyl-3-phosphatidylserine (DPPS); palmitoyloleoyl phosphatidylethanolamine (POPE); and 1,2-dioleoyl-3-phosphatidylserine (DOPS).

24. The fusogenic liposome of claim 23, wherein said phospholipid is selected from the group consisting of DOPC, POPC, DMPC, DPPC, DOPE, POPE, DSPE, DMPE and DPPE.

25. The fusogenic liposome of claim 1, wherein the stabilizing moiety is PEG of molecular weight of from about 106 Da to about 4 kDa.

26. The fusogenic liposome of claim 25, wherein PEG is of molecular weight of about 2 kDa.

27. The fusogenic liposome of claim 1, wherein said stabilizing moiety is connected to at least one of said lipid molecules via a cleavable peptide linker.

28. The fusogenic liposome of claim 5, wherein said first functional group of the specific binding pair is capable of forming a covalent bond with said complementary second functional group of said binding pair.

29. The fusogenic liposome of claim 28, wherein said first functional group of the specific binding pair is capable of forming a covalent bond with said complementary second functional group of said binding pair via a click chemistry reaction.

30. The fusogenic liposome of claim 28, wherein: i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa; iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or a derivative thereof, and the second functional group of said binding pair is amine, or vice versa.

31. The fusogenic liposome of claim 30, wherein the specific binding pair is alkyne-azide.

32. The fusogenic liposome of claim 5, wherein said first functional group of the specific binding pair is capable of forming a non-covalent bond with said complementary second functional group of said binding pair.

33. The fusogenic liposome of claim 32, wherein the first functional group of the specific binding pair is biotin, and the second functional group of said binding pair selected from the group consisting of a biotin-binding peptide or biotin-binding protein, or vice versa.

34. The fusogenic liposome of claim 33, wherein said biotin-binding protein is selected from the group consisting of avidin, streptavidin and an anti-biotin antibody.

35. The fusogenic liposome of claim 34, wherein said biotin-binding peptide is selected from the group consisting of AEGEFCSWAPPKASCGDPAK (SEQ ID NO: 1), CSWRPPFRAVC (SEQ ID NO: 2), CSWAPPFKASC (SEQ ID NO: 3), and CNWTPPFKTRC (SEQ ID NO: 4).

36. The fusogenic liposome of claim 1, wherein the fusogenic liposome further comprises cholesterol (CHO) or its derivatives.

37. The fusogenic liposome of claim 1, wherein the fusogenic liposome comprises DOPC:DOTAP:DSPE-PEG2K:DOPE or DOPC:DOTAP:DSPE-PEG2K, and optionally cholesterol, wherein PEG2K represents PEG having a molecular weight of about 2 kDa, and the relative molar amount of DOPC is up to about 80%, the relative molar amount of DOTAP is up to about 80%, the relative molar amount of DSPE-PEG2K is up to about 20%, the relative molar amount of DOPE is up to about 20%, the relative molar amount of cholesterol is up to about 40%.

38. The fusogenic liposome of claim 37, wherein the fusogenic liposome comprises:
(iii) DOPC:DOTAP:DSPE-PEG2K:DOPE in the molar ratio 52.5:35:0.6:10, 52.5:35:1.25:10, 52.5:35:2.5:10, 52.5:35:5:10, 52.5:35:0.6:5, 52.5:35:1.25:5, 52.5:35:2.5:5, 52.5:35:5:5, 65:20:5:10, 50:35:5:10, 52.5:35:1.25:7, 52.5:35:1.25:5, or 52.5:35:2.5:7; or
(iv) DOPC:DOTAP:DSPE-PEG2K, in the molar ratio 52.5:35:0.6, 52.5:35:1.25, 52.5:35:2.5, 52.5:35:5, 52.5:35:0.6, 52.5:35:1.25, 52.5:35:2.5, 52.5:35:5, 65:20:5, 50:35:5, 52.5:35:1.25, 52.5:35:1.25, or 52.5:35:2.5.

39. The fusogenic liposome of claim 38, wherein the fusogenic liposome comprises DOPC:DOTAP:DSPE-PEG2K:DOPE in the molar ratio 52.5:35:2.5:5; or DOPC:DOTAP:DSPE-PEG2K, in the molar ratio 52.5:35:2.5.

40. The fusogenic liposome of claim 1, wherein the melting temperature (Tm) of the liposome is below 45° C., at which the fusogenic liposome is maintained at a non-crystalline transition phase thereby providing membrane fluidity required for fusion of liposome with cell membranes.

41. The fusogenic liposome of claim 1, wherein the fusogenic liposome has a size of up to 200 nm, e.g., from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, or about 100 nm.

42. A method for selectively detecting cancer cells, comprising contacting said cancer cells with a fusogenic liposome of claim 1, and in case said detectable agent is a fluorescent probe, detecting said fluorescent probe by illuminating the cell with light having a wave length that is absorbed by the fluorescent probe and detecting light emitted from the excited fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent.

43. The method of claim 42, wherein selective detection of cancer cells in a cancer patient indicates responsiveness of said cancer patient to treatment of cancer with a cancer drug comprised in a nanoparticle.

44. The method of claim 43, wherein the cancer patient determined as responsive is treated with said nanoparticle comprising said cancer drug.

45. A method for selectively detecting cancer cells, comprising
a. contacting said cancer cells with a functionalised fusogenic liposome of claim 5;
b. contacting said cancer cells with a detectable agent selected from the group consisting of a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and
c. in case said detectable agent is a fluorescent probe, detecting said fluorescent probe by illuminating the cell with light having a wave length that is absorbed by the fluorescent probe and detecting light emitted from the fluorescent probe; in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby selectively detecting said cancer cells.

46. The method of claim 45, wherein said detectable agent is (a) a fluorescent probe selected from the group consisting of cy3, cy5, cy5.5, cy7 cy9, FITC, fluorescein, alexa fluor 790, alexa fluor 750, alexa fluor 700, alexa fluor 680, alexa fluor 660, alexa fluor 647, alexa fluor 633, alexa fluor 594, Qdots ranging 585 nm to 800 nm, fluorescent protoporphyrin oligomers, and isocyanine green (ICG); or (b) an activatable fluorescent probe selected from the group consisting of fluorescein analogs, coumarin analogs, CFSE (5(6)-Carboxyfluorescein diacetate N-succinimidyl ester), rhodamine analogs, curcuminoid difluoroboron-based tumor-targeting γ-glutamyltranspeptidase (GGT)-activatable) fluorescent probe (Glu-DFB), and an indocyanine analog.

47. The method of claim 45, wherein selective detection of cancer cells in a cancer patient indicates responsiveness of said cancer patient to treatment of cancer with a cancer drug comprised in a nanoparticle.

48. The method of claim 47, wherein the cancer patient determined as responsive is treated with said nanoparticle comprising said cancer drug.

49. A method for treating cancer by fluorescence-guided surgery or targeted radiotherapy, said method comprising the method of claim 42 and removing the tumor containing the cancer cells.

50. The method of claim 42, wherein said cancer patient is undergoing imaging of tumors, such as skin cancer, or whole body imaging, said method comprising systemically administering or topically applying the fusogenic liposome, and optionally the functionalized detectable agent; and, in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent, thereby defining tumor location and margins.

51. A method for treating cancer comprising the method of claim 50 and removing the tumor containing the cancer cells.

52. The method of claim 42, for selectively detecting cancer cells ex situ in a tissue or a blood-derived fraction removed from the cancer patient in need thereof, said method comprising systemically administering or applying the fusogenic liposome, and optionally the functionalized fluorescent probe, to the tissue or blood-derived fraction and detecting said fluorescent probe by illuminating the tissue or blood-derived fraction and detecting light emitted from the fluorescent probe, thereby selectively detecting said cancer cells.

53. The method of claim 52, wherein said tissue is skin, said cancer patient is undergoing surgery for removal of skin cancer (Mohs surgery), and said surgery is repeated until the skin tissue has no detectable cancer cells.

54. The method of claim 52, for selectively detecting circulating tumour cells (CTCs) in said blood-derived fraction.

55. The method of claim 42, wherein said cancer is selected from the group consisting of breast cancer, such as triple-negative breast cancer, melanoma, lung cancer, thyroid cancer and prostate cancer.

56. The method of claim 42, for selectively detecting cancer cells and treating cancer, said method comprising systemically administering or topically applying the fusogenic liposome and optionally the functionalized detectable agent, wherein the fusogenic liposome comprises said detectable agent and a cytotoxic agent and/or immune system activating agent; in case said detectable agent is an activatable fluorescent probe or fluorescent probe, detecting said fluorescent probe by illuminating an area of the skin or the whole body and detecting light emitted from the fluorescent probe, in case said detectable agent is a contrast agent, rendering an image by analysing changes in signal intensity by the means of an MRI, CT or PET device, or in case said fusogenic liposome comprises both a fluorescent probe and a contrast agent, detecting both said fluorescent probe and said contrast agent; and optionally monitoring treatment response by repeatedly detecting said detectable agent over time.

57. A kit comprising:
a. a first container comprising a fusogenic liposome comprising a lipid bilayer comprising a plurality of lipid molecules having 14 to 24 carbon atoms, and at least one of said lipid molecules further comprises a cationic group, a cationic natural or synthetic polymer, a cationic amino sugar, a cationic polyamino acid or an amphiphilic cancer-cell binding peptide;
at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide; and
wherein at least one of said lipid molecules is functionalised with a first functional group of a specific binding pair capable of binding to a complementary second functional group of said binding pair;
b. a second container comprising a detectable agent selected from the group consisting of a fluorescent probe and a contrast agent for magnetic resonance imaging (MRI), computed tomography (CT) or positron emission tomography (PET), wherein said detectable agent is functionalized with a complementary second functional group of the binding pair capable of binding to said first functional group of said lipid molecules; and
c. a pamphlet with instructions for a method for selectively detecting cancer cells comprising administering to a cancer patient the fusogenic liposome of (a) and subsequently the detectable agent of (b).


SEQUENCE LISTING (4 sequences)

SEQ ID NO: 1 - protein, 20 aa, Artificial Sequence
  synthetic
AEGEFCSWAPPKASCGDPAK

SEQ ID NO: 2 - protein, 11 aa, Artificial Sequence
  synthetic
CSWRPPFRAVC

SEQ ID NO: 3 - protein, 11 aa, Artificial Sequence
  synthetic
CSWAPPFKASC

SEQ ID NO: 4 - protein, 11 aa, Artificial Sequence
  synthetic
CNWTPPFKTRC